(19) United States
(12) Patent Application Publication
(10) Pub. No.: US 2011/0034975 A1
(43) Pub. Date: Feb. 10, 2011

(54) METHODS AND APPARATUS FOR REPAIRING AND/OR REPLACING INTERVERTEBRAL DISC COMPONENTS AND PROMOTING HEALING

(76) Inventor: Bret A. Ferree, Cincinnati, OH (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/855,971

(22) Filed: Aug. 13, 2010

Related U.S. Application Data
(63) Continuation-in-part of application No. 11/805,677, filed on May 23, 2007.
Application No. 11/805,677 is a continuation-in-part of application No. 12/263,753, filed on Nov. 3, 2008.
Application No. 12/263,753 is a continuation-in-part of application No. 11/811,751, filed on Jun. 12, 2007.
Application No. 11/811,751 is a continuation-in-part of application No. 11/946,001, filed on Nov. 27, 2007.
Application No. 11/946,001 is a continuation-in-part of application No. PCT/US2009/065954, filed on Nov. 25, 2009.
(60) Provisional application No. 60/808,795, filed on May 26, 2006.
(60) Provisional application No. 60/813,232, filed on Jun. 13, 2006.
(60) Provisional application No. 60/847,649, filed on Sep. 26, 2006.
(60) Provisional application No. 60/984,657, filed on Nov. 1, 2007.
(60) Provisional application No. 60/861,499, filed on Nov. 28, 2006.
(60) Provisional application No. 61/233,986, filed on Aug. 14, 2009.
(60) Provisional application No. 61/118,246, filed on Nov. 26, 2008.

(51) Int. Cl. A61F 7/00; A61F 2/44

(52) U.S. Cl. 607/105; 623/17.16

(57) ABSTRACT

Disclosed embodiments provide for treatment of the anulus fibrosus (AF) and intevertebral disc (IVD), including herniated discs, anular tears of the disc, or disc degeneration, while enabling surgeons to contain blood, fluid, proteins, or other materials that are placed into or migrate into or near defective regions of the spine. The invention also concentrates mesenchymal stem cells (MSCs) in the surgical area and increases the area of bone to be fused. Other aspects of the invention may be used to raise the temperature of tissues, including surgical tissues, to stimulate inflammation, thereby stimulating tissue healing. According to these embodiments, the invention places a heating element below the skin and adjacent to the tissue to stimulate the healing thereof.

### REFERENCE TO RELATED APPLICATIONS

This application is a continuation-in-part of U.S. Ser. No. 11/805,677, filed May 23, 2007, which claims priority from U.S. Provisional Patent Application Ser. No. 60/808,795, filed May 26, 2006.

This application is also a continuation-in-part of U.S. patent application Ser. No. 12/263,753, filed Nov. 3, 2008, which is a continuation-in-part of U.S. patent application Ser. No. 11/811,751, filed Jun. 12, 2007, which claims priority from U.S. Provisional Patent Application Ser. Nos. 60/813,232, filed Jun. 13, 2006 and 60/847,649, filed Sep. 26, 2006.

U.S. patent application Ser. No. 12/263,753 claims priority from U.S. Provisional Patent Application Ser. No. 60/984,657, filed Nov. 1, 2007.

This application is also a continuation-in-part of PCT/US2009/065954, filed Nov. 25, 2009, which claims priority from U.S. Provisional Patent Application Ser. No. 61/118,246, filed Nov. 26, 2008.

This application is also a continuation-in-part of U.S. Ser. No. 11/946,001, filed Nov. 27, 2007, which claims priority from U.S. Provisional Patent Application Ser. No. 60/861,499, filed Nov. 28, 2006.

This application claims priority from U.S. Provisional Patent Application Ser. No. 61/233,986, filed Aug. 14, 2009.

The entire content of each application is incorporated herein by reference.

### FIELD OF THE INVENTION

This invention relates generally to the treatment of intervertebral disc herniation and degenerative disc disease and, in particular, to apparatus and methods for fortifying, sealing and/or replacing disc components such as the anulus fibrosis.

### BACKGROUND OF THE INVENTION

The human intervertebral disc is an oval to kidney bean-shaped structure of variable size depending on the location in the spine. The outer portion of the disc is known as the anulus fibrosus (AF, also known as the “anulus fibrosis”). The anulus fibrosus (AF) is made of ten to twenty collagen fiber lamellae. The collagen fibers within a lamella are parallel. Successive lamellae are oriented in alternating directions. About 48 percent of the lamellae are incomplete, but this value varies based upon location and increases with age. On average, the lamellae lie at an angle of sixty degrees with respect to the vertebral axis line, but this too varies depending upon location. The orientation serves to control vertebral motion (one half of the bands tighten to check motion when the vertebra above or below the disc are turned in either direction).

The anulus fibrosus contains the nucleus pulposus (NP). The nucleus pulposus serves to transmit and dampen axial loads. A high water content (approximately 70-80 percent) assists the nucleus in this function. The water content has a diurnal variation. The nucleus imbibes water while a person lies recumbent. Nuclear material removed from the body and placed into water will imbibe water swelling to several times its normal size. Activity squeezes fluid from the disc. The nucleus comprises roughly 50 percent of the entire disc. The nucleus contains cells (chondrocytes and fibrocytes) and proteoglycans (chondroitin sulfate and keratin sulfate). The cell density in the nucleus is on the order of 4,000 cells per microliter.

The intervertebral disc changes or “degenerates” with age. As a person ages, the water content of the disc falls from approximately 85 percent at birth to approximately 70 percent in the elderly. The ratio of chondroitin sulfate to keratin sulfate decreases with age, while the ratio of chondroitin 6 sulfate to chondroitin 4 sulfate increases with age. The distinction between the anulus and the nucleus decreases with age. Generally disc degeneration is painless.

Premature or accelerated disc degeneration is known as degenerative disc disease. A large portion of patients suffering from chronic low back pain are thought to have this condition. As the disc degenerates, the nucleus and anulus functions are compromised. The nucleus becomes thinner and less able to handle compression loads. The anulus fibers become redundant as the nucleus shrinks. The redundant anular fibers are less effective in controlling vertebral motion. This disc pathology can result in: 1) bulging of the anulus into the spinal cord or nerves; 2) narrowing of the space between the vertebra where the nerves exit; 3) tears of the anulus as abnormal loads are transmitted to the anulus and the anulus is subjected to excessive motion between vertebra; and 4) disc herniation or extrusion of the nucleus through complete anular tears.

Current surgical treatments for disc degeneration are destructive. One group of procedures, which includes lumbar discectomy, removes the nucleus or a portion of the nucleus. A second group of procedures destroy nuclear material. This group includes Chymopapin (an enzyme) injection, laser discectomy, and thermal therapy (heat treatment to denature proteins). The first two groups of procedures compromise the treated disc. A third group, which includes spinal fusion procedures, either removes the disc or the disc's function by connecting two or more vertebra together with bone. Fusion procedures transmit additional stress to the adjacent discs, which results in premature disc degeneration of the adjacent discs. These destructive procedures lead to acceleration of disc degeneration.

Prosthetic disc replacement offers many advantages. The prosthetic disc attempts to eliminate a patient's pain while preserving the disc's function. Current prosthetic disc implants either replace the nucleus or replace both the nucleus and the anulus. Both types of current procedures remove the degenerated disc component to allow room for the prosthetic component. Although the use of resilient materials has been proposed, the need remains for further improvements in the way in which prosthetic components are incorporated into the disc space to ensure strength and longevity. Such improvements are necessary, since the prosthesis may be subjected to 100,000,000 compression cycles over the life of the implant.

Current nucleus replacements (NRs) may cause lower back pain if too much pressure is applied to the anulus fibrosus. As discussed in co-pending U.S. Pat. Nos. 6,878,167 and 7,201,774, the content of each being expressly incorporated herein by reference in their entirety, the posterior portion of the anulus fibrosus has abundant pain fibers.

Herniated nucleus pulposus (HNP) occurs from tears in the anulus fibrosus. The herniated nucleus pulposus often allies pressure on the nerves or spinal cord. Compressed nerves cause back and leg or arm pain. Although a patient's symptoms result primarily from pressure by the nucleus pulposus, the primary pathology lies in the anulus fibrosus.

Surgery for herniated nucleus pulposus, known as microlumbar discectomy (MLD), only addresses the nucleus pulposus. The opening in the anulus fibrosus is enlarged during surgery, further weakening the anulus fibrosus. Surgeons also remove generous amounts of the nucleus pulposus to reduce the risk of extruding additional pieces of nucleus pulposus through the defect in the anulus fibrosus. Although microlumbar discectomy decreases or eliminates a patient's leg or arm pain, the procedure damages weakened discs.

### SUMMARY OF THE INVENTION

The invention broadly facilitates reconstruction of the anulus fibrosus (AF). Such reconstruction seals the intevertebral disc (IVD). The invention may also be used in the treatment of herniated discs, anular tears of the disc, or disc degeneration, while enabling surgeons to contain blood, fluid, proteins, or other materials that are placed into or migrate into or near defective regions of the spine. Containment of such fluids or materials prevents hematomas (collection of blood within the body, but outside of blood vessels). Hematomas may compress structures that lie adjacent to the spine, such as the esophagus and the spinal cord, which can cause death or paralysis. Hematomas also cause adhesions and may cause pain. Adhesions may cause pain and increase the risk of revision surgery. Containment of bone growth materials, such as bone morphogenic protein (BMP), autograft or allograft bone, demineralized bone matrix, synthetic bone substances, or other such material facilitates spinal fusion and helps prevent post-operative complications. For example, BMP that leaks from the disc space may cause life-threatening swelling. The invention also concentrates mesenchymal stem cells (MSCs) in the surgical area. Lastly, the invention increases the area of bone to be fused. The methods and apparatus may be used to treat discs throughout the spine including the cervical, thoracic, and lumbar spines of humans and animals. For example, the methods and apparatus may be used in surgeries on the anterior, lateral, or posterior portions of the spine. The methods and apparatus may be used in other bones or tissues of the body. For example, the invention may be used to treat avascular necrosis of the hip.

The invention also enables surgeons to reconstruct the anulus fibrosus and replace or augment the nucleus pulposus. Novel nucleus replacements (NR) or total disc replacements (TDR) may be added to the disc. Anulus reconstruction prevents extrusion of the nucleus replacements through holes in the anulus fibrosus. The nucleus replacements and the anulus fibrosus reconstruction prevent excessive pressure on the anulus fibrosus that may cause back or leg pain. The nucleus replacements may be made of natural or synthetic materials. Synthetic nucleus replacements may be made of, but are not limited to, polymers including polyurethane, silicon, hydrogel, or other elastomers. Total disc replacements may be made of titanium, chrome cobalt, or other material.

A spinal repair system according to the invention comprises flexible longitudinal fixation components adapted for placement in bone or through portions of the AF with intact fibers, a micro-porous mesh reinforcement and anti-adhesion component for placement over portions of IVDs and vertebrae. The flexible longitudinal fixation component may anchored to one of the upper and lower vertebral bodies. Tension on the flexible fixation components presses the compliant anti-adhesion component against the vertebrae and the IVDs, thus preventing blood, fluids, proteins, or other materials from passing from the spine into the tissues that surround the spine.

Other aspects of the invention may be used to raise the temperature of tissues, including surgical tissues, to stimulate inflammation, thereby stimulating tissue healing. According to these embodiments, the invention places a heating element below the skin and adjacent to the tissue being stimulated or treated. In contrast to existing techniques, the heat from prior art external devices is concentrated on the skin and the tissues near the skin rather than on the deeper tissues. Thus, peripheral heating elements may increase inflammation and attract MSCs to the peripheral tissues rather than to the deeper tissues.

### BRIEF DESCRIPTION OF THE DRAWINGS

FIG. 1A shows an anterior view of a portion of the spine and an embodiment of the invention including an anti-adhesion component flexible longitudinal fixation components;

FIG. 1B shows a lateral view of a partial sagittal cross section of a portion of the spine;

FIG. 2A shows an anterior view of a portion of the spine and an alternative embodiment of the invention drawn in FIG. 1A;

FIG. 2B shows an anterior view of the spinal segment and the embodiment of the invention drawn in FIG. 2A;

FIG. 2C shows an anterior view of a portion of the spine and an alternative configuration;

FIG. 3 shows an anterior view of a portion of the spine and a further alternative configuration;

FIG. 4 shows an anterior view of a portion of the spine and the embodiment of the invention drawn in FIG. 2A;

FIG. 5A shows an anterior view of a portion of the spine wherein the device drawn in FIG. 2A was applied across two IVDs;

FIG. 5B shows an anterior view of a portion of the spine and an alternative configuration;

FIG. 6 shows an anterior view of a portion of the spine, the embodiment of the invention drawn in FIG. 5A and a prior art plate and screws;

FIG. 7 shows an anterior view of a portion of the spine, the embodiment of the invention drawn in FIG. 5A and two prior art plates and screws;

FIG. 8 shows an anterior view of an alternative embodiment of the plates and screws drawn in FIGS. 6 and 7

FIG. 9 shows an anterior view of an alternative embodiment of the invention drawn in FIG. 8;

FIG. 10A shows an anterior view of a portion of the spine and an alternative embodiment of the invention drawn in FIG. 1A;

FIG. 10B is an anterior view of the portion of the spine, the embodiment of the invention drawn in FIG. 10A and an anti-adhesion cover;

FIG. 11A is an anterior view of a portion of the spine;

FIG. 11B is an anterior view of the portion of the spine and the embodiment of the invention drawn in FIG. 11A;

FIG. 11C is a lateral view of a partial sagittal cross section of a portion of the spine and the embodiment of the invention drawn in FIG. 11A;

FIG. 12A is an anterior view of an anti-adhesion cover and prior art methods of fastening such cover to the spine or other area of the body;

FIG. 12B is an anterior view of the embodiment of the invention;

FIG. 12C is an anterior view of the embodiment of the invention drawn in FIG. 1A;

FIG. 12D is an anterior view of the embodiment of the invention drawn in FIG. 2A;

FIG. 13A is an anterior view of a portion of the spine and an alternative embodiment of the invention drawn in FIG. 6;

FIG. 13B is a lateral view of a partial sagittal cross section of the portion of the spine and embodiment of the invention drawn in FIG. 13B;

FIG. 13C is a view of an axial cross section of an IVD and the embodiment of the invention drawn in FIG. 13B;

FIG. 13D is an anterior view of a portion of the spine and the embodiment of the invention drawn in FIG. 13A;

FIG. 13E is an anterior view of a portion of the spine and the embodiment of the invention drawn in FIG. 13D;

FIG. 13F is a lateral view of a partial sagittal cross section of the spine and the embodiment of the invention drawn in FIG. 13E;

FIG. 14A is an anterior view of a partial coronal cross section of the proximal end of the femur and an alternative embodiment of the invention;

FIG. 14B is an anterior view of a partial coronal cross section of the proximal end of the femur and an alternative embodiment of the invention drawn in FIG. 14A;

FIG. 15A is an anterior view of an alternative anti-adhesion cover of an alternative embodiment of the invention drawn in FIG. 4;

FIG. 15B is an anterior view of a second anti-adhesion cover used in the embodiment of the invention drawn in FIG. 15A;

FIG. 15C is an anterior view of a spinal segment and the anti-adhesion covers drawn in FIGS. 15A and 15B;

FIG. 16 is an anterior view of a spinal segment and an alternative embodiment of the invention drawn in FIG. 15C;

FIG. 17 is an anterior view of an alternative embodiment of the invention drawn in FIG. 4;

FIG. 18 is an anterior view of the distal portion of an alternative embodiment of the invention drawn in FIG. 17; and

FIG. 19 is schematic of a resistive heating element applicable to the invention.

### DETAILED DESCRIPTION OF THE INVENTION

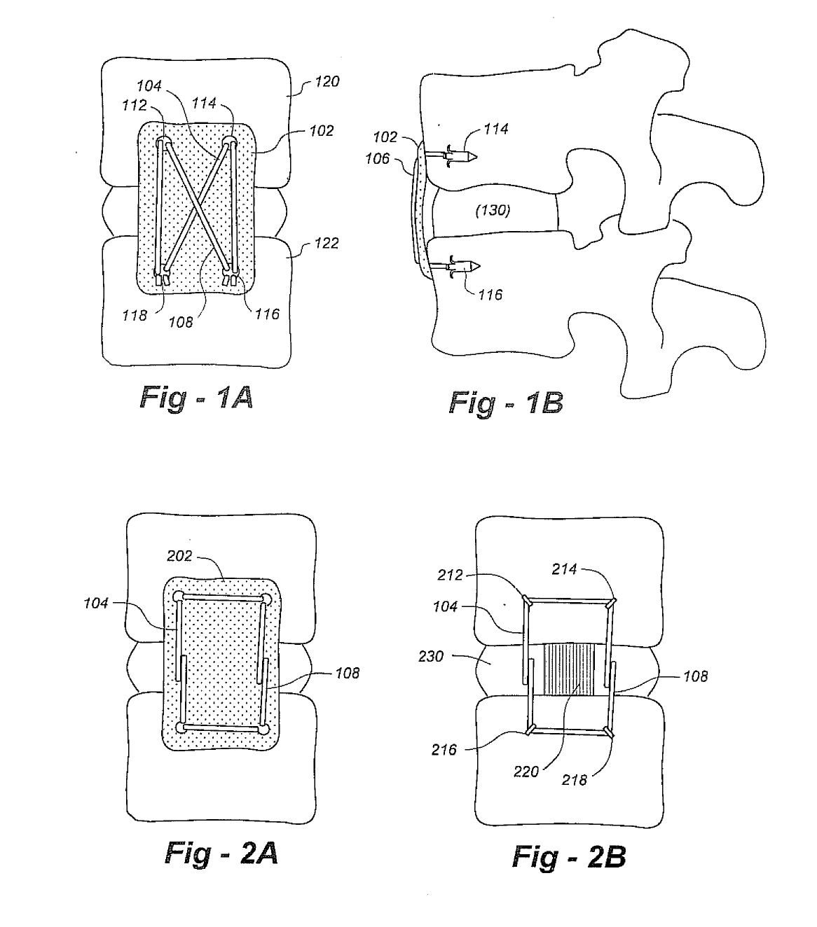

FIG. 1A is an anterior view of a portion of the spine and an embodiment of the invention including an anti-adhesion component 102 flexible longitudinal fixation components 104, 108. FIG. 1B is a lateral view of a partial sagittal cross section of a portion of the spine, the embodiment of the invention drawn in FIG. 1A and an intra-discal cage 130.

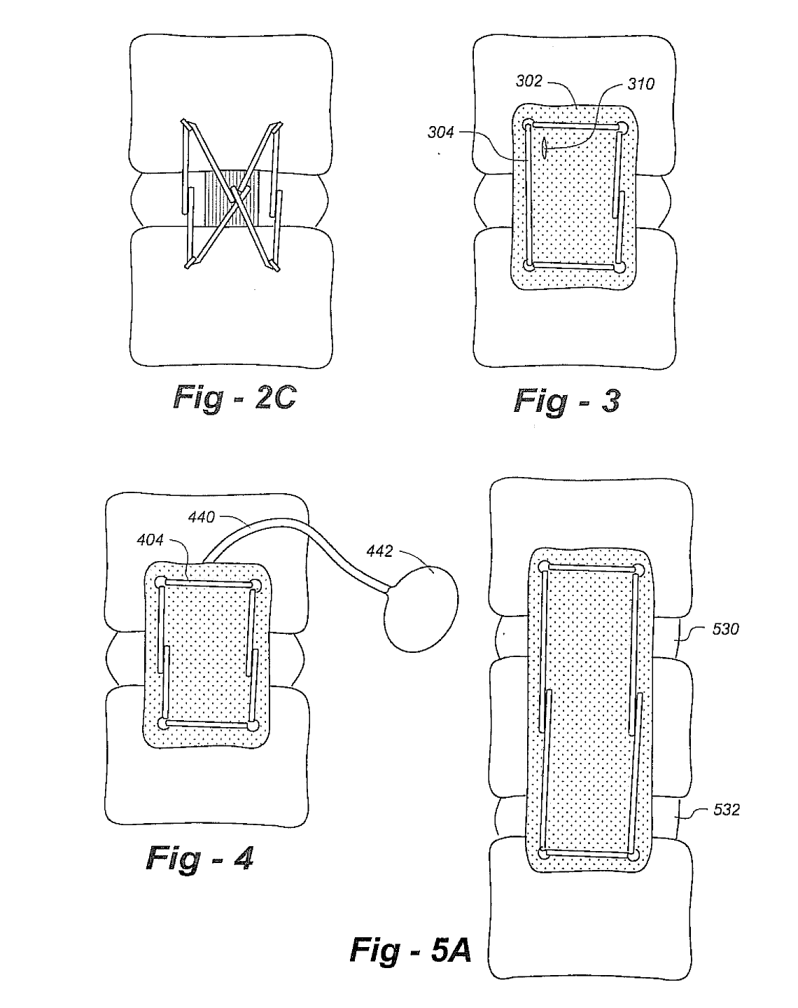

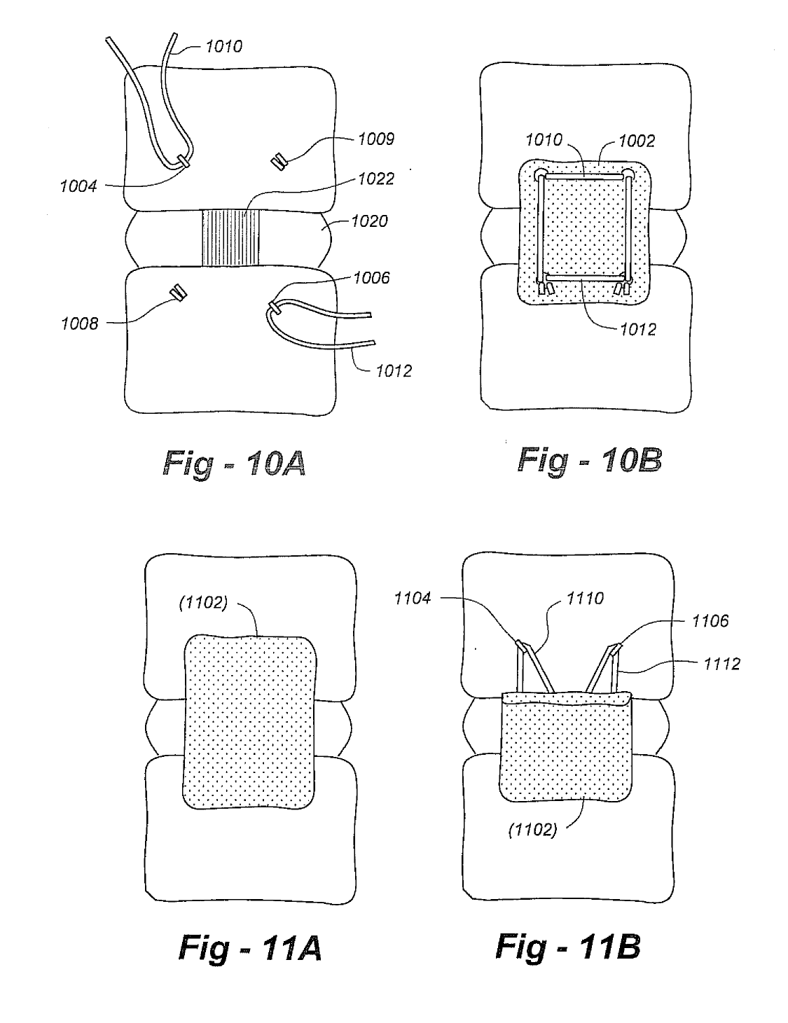

This embodiment is related to the invention depicted in FIG. 8G of co-pending provisional patent application U.S. Ser. No. 61/118,246, FIGS. 2A, 8A-8G of co-pending patent application U.S. application Ser. No. 11/805,677 and FIGS. 10A and 10B of my co-pending patent application U.S. application Ser. No. 11/946,001.

The anti-adhesion component 102 in this and other embodiments of the invention is preferably made of synthetic micro-porous material, allograft tissue or xenograft tissue. The pores of the material are preferably less than 4 to 5 microns in width and prevent blood, fluids, and proteins from migrating through the material, which might occur if the component 102 were made of a mesh material having larger pore sizes. For example, the component could be made of expanded polytetrafluoroethylene (ePTFE) allograft fascia, or porcine intestinal submucosa, and may be 5 to 25 millimeters wide, 5 to 50 millimeters long and 0.2 to 4 millimeters thick. The anti-adhesion component may be larger or smaller in alternative embodiments of the invention.

The flexible longitudinal fixation components 104, 108 taught in this and other embodiments of the invention are preferably made of high tensile strength multi-filament or braided polyester. For example, the flexible longitudinal fixation component could be made of #2 to #5 sized Fiberwire (Arthrex, Naples, Fla., USA), Orthocord (DePuy Orthopaedics, Warsaw, Ind., USA), suture from Tornier (Edina, Minn., USA), nylon or other type or size suture material. The flexible longitudinal components are preferably in the range of 20 to 60 millimeters long.

The flexible longitudinal components are held in position with anchors 112, 114, 116, 118. These anchors are preferably “push-in” anchors with an expandable or deployable component. In FIGS. 1A and 1B, it is assumed that anchors 112, 114 are of the non-locking type, allowing elements 108, 104 to be respectively threaded therethrough, whereas anchors 118, 116 are of the locking type, allowing the ends of elements 104, 108 to be captured as shown, preferably following tensioning. It will be appreciated, however, that other arrangements of non-locking and/or locking anchors may be implemented, as shown in FIGS. 10A and 10B, for example.

Push-in anchors have appendages that expand away from the shaft of the anchor after the anchor is inserted into bone. Alternatively, push-in anchors may expand in a radial direction after the anchors are inserted into bone. Push-in anchors do not have threads and are not screwed into bone. Push-in anchors are generally impacted into bone or holes drilled into bone. Examples of nonscrew-in or push-in anchors include Piton (Tornier, Edina Minn.), Impact, UltraFix RC, Ultrafix MiniMite anchors (Conmed, Largo Fla.), Bioknotless, GII, Versalok, Micro, and Super anchors (DePuy Mitek), (Raynham Mass.), Bio-SutureTak (Arthrex Naples, Fla.), and Collared Harpoon and Umbrella Cancellous Harpoon (Arthrotek, Warsaw, Ind.). The anchors are preferably made of titanium or other MRI compatible material. Alternatively, the anchors could be made of plastic such as Deiron, or a bioresorbable such as polylactic acid (PLA), polyglycolic acid (PGA), poly (ortho esters), poly(glycolide-co-trimethylene carbonate), poly-L-lactide-co-6-caprolactone, polyanhydrides, poly-n-dioxanone, poly(PHB-hydroxyvaleric acid), or combinations thereof.

The anti-adhesion cover 102 in FIGS. 1A and 1B is fastened to the vertebra cranial 120 and caudal 122 to fortify, seal and/or replacing disc components such as the anulus fibrosis AF. The cover may also be fastened or anchored to intevertebral disc (IVD) components, including the AF. The invention may be used to cover and protect a naturally occurring defect in the AF or a portion of the AF that was incised or excised to create a defective area associated with the insertion of an intradiscal component such as a cage filled with bone growth material.

The anti-adhesion cover is preferably impervious to fluids, such as blood, and proteins such as BMP. The anti-adhesion cover and the fixation components prevent or limit the egress of blood, fluids, proteins, cells, or other materials into and out of the IVD. The invention prevents adhesions by preventing the growth of connective tissue across the anti-adhesion cover and into or onto the IVD. The invention also prevents blood from leaking from the IVD and into the tissues surrounding the IVD. Such blood collections, know as hematomas, cause adhesions. The invention also keeps added materials such as BMP from leaking from the IVD. BMP that leaks from the IVD may cause life-threatening swelling of the tissues of the neck.

The anti-adhesion cover is preferably fastened to the spine with the apparatus and methods taught in FIGS. 36A and 36B and described in my co-pending U.S. patent application Ser. No. 12/173,775. As shown in FIGS. 1A and 1B the preferred fixation system includes four suture anchors and two flexible longitudinal fixation elements. The flexible longitudinal elements are preferably coupled or lockingly fastened to the suture anchors. The flexible longitudinal elements are captured by a locking mechanism in the anchors in the vertebra caudal to the disc. For example, Piton anchors may be used to capture the ends of the flexible longitudinal elements.

The flexible longitudinal fixation elements and preferably the suture anchors were passed through four openings in the anti-adhesion cover. The flexible longitudinal fixation components press the compliant anti-adhesion cover to the spine, thus forming a seal between the anti-adhesion cover and the spine. The invention enables sealing of irregular surfaces that are often found on the spine.

FIG. 2A is an anterior view of a portion of the spine and an alternative embodiment of the invention drawn in FIG. 1A. The ends of two flexible longitudinal fixation elements 104, 108 were welded (Axya Medical, Beverly Mass.) to each other over the anti-adhesion cover 202 in the configuration shown. Alternatively, preferably “knotless” methods or devices may be used to fasten the ends of the flexible longitudinal fixation components to each other. For example, additional components could be welded or crimped around the ends of the flexible fixation members. Four “push-in” anchors are preferably used to anchor the flexible longitudinal fixation elements to the vertebrae using the previously described method.

FIG. 2B is an anterior view of the spinal segment and the embodiment of the invention drawn in FIG. 2A. The drawing illustrates the configuration of the four anchors 212, 214, 216, 218 and the two flexible longitudinal fixation elements 104, 108 drawn in FIG. 2A. The anti-adhesion cover was not drawn in the figure to better illustrate the position of the vertical arms of the flexible longitudinal fixation elements. An intra-discal cage 220 is seen within the IVD 230. A rectangular shaped portion of the AF was surgically removed (anulotomy) to enable insertion of the cage in the IVD. The vertical arms of the flexible longitudinal fixation elements lie over intact AF tissue on either side of the aperture in the AF. Such position of the elongate elements presses the anti-adhesion cover against the AF tissue and the vertebrae that surround the aperture in the AF, thus preventing blood, fluid, BMP, or other materials from contacting the tissues adjacent to the spine.

FIG. 2C is an anterior view of a portion of the spine and an alternative configuration wherein the ends of the two flexible longitudinal fixation elements were welded over the intra-discal device in the configuration illustrated. The anti-adhesion cover, which normally lies under the flexible longitudinal fixation elements was not drawn to better illustrate the position of such flexible members and the intra-discal device.

FIG. 3 is an anterior view of a portion of the spine and a further alternative configuration wherein the ends of a single flexible longitudinal fixation element 304 were welded or otherwise fastened together after applying tension on the ends of the element. Two suture anchors above and below the IVD, fasten the flexible longitudinal element to the spine. A small slit 310 is seen in the anti-adhesion cover 302. Such slit enables egress of small amounts of fluid, blood or other material from the IVD. However, the slit prevents accumulation of too much blood or other material within the IVD. The slit is preferably 1 to 6 millimeters in length. Two, three, four or more slits or other openings 0.5, 0.6, 0.7, 0.8, 0.9, 6.1, 6.2, 6.3, 6.4, smaller than 0.5 or larger than 6.4 millimeters in length may also be used in accordance with the invention. Such openings may preferably be located over each disc that lies under the cover. In an alternative embodiment of the invention, the anti-adhesion cover could be designed to tear, if pressure under the cover exceeds a predetermined level.

FIG. 4 is an anterior view of a portion of the spine and the embodiment of the invention drawn in FIG. 2A. The catheter portion 440 of a surgical drain 442 extends under the anti-adhesion cover 402 and under the horizontal section of the upper flexible longitudinal component 404. The drain prevents accumulation of excessive blood or fluid within the IVD. The drain preferably aspirates bone marrow contents, including blood and mesenchymal stem cells (MSCs) from perforations through the vertebral endplates. The drain removes excess blood from the disc space. However, aspirated MSCs bind to bone growth materials placed into the disc space, thus concentrating the MSCs in the disc space.

The invention pulls MSC rich blood from the marrow of the vertebrae and removes MSC poor blood from the area under the anti-adhesion cover. Such invention increases the number and the concentration of MSCs under the anti-adhesion cover. The seal between the anti-adhesion cover and the spine and the drain tip placed under the anti-adhesion cover and the flexible longitudinal fixation element enables aspiration of bone marrow contents, in-situ, from the vertebrae.

The tip of the drain preferably extends into the disc space and most preferably extends into bone growth material used for fusion procedures. The tip of the drain could extend through and opening in the side of an interbody fusion cage. Alternatively, however, the tip of the drain could be placed into the disc space and preferably into or onto or near bone growth material without including the anti-adhesion cover and the seal provided by such cover.

The proximal portion of the catheter preferably extends through patient's skin to enable periodic emptying of the reservoir and removal of the drain. The drain is preferably pulled from the surgical site 24 to 48 hours after surgery. The drain may be removed sooner or later in other embodiments of the invention. The catheter of the drain preferably has an internal diameter of 1 to 3 millimeters, an external diameter of 1.2 to 5 millimeters and a length of 15 to 80 millimeters. The reservoir preferably holds 10 to 50 milliliters of fluid.

The drain is preferably made of flexible, biocompatible materials such as polyurethane, polypropylene, silicon, or other such material. The drain may preferably apply intermittent suction. For example, the drain my apply suction 10, 15, 20, 25, 30, 35, 40, less than 10, or more than 40 minutes each hour. Alternatively, the magnitude of the suction could vary with time. For example, the magnitude of the suction could be increased from baseline suction 10, 15, 20, 25, 30, 35, 40, less than 10 or more than 40 minutes each hour. Alternatively, the suction could be constant while the drain tip of the drain is under the anti-adhesion cover. Alternatively, an elongate device such as a sheet of silastic could be inserted under the anti-adhesion cover and a flexible longitudinal fixation element while tension is applied to the ends of the flexible longitudinal fixation element and the ends of such element are welded or otherwise fastened to each other.

The elongate device is then removed leaving a small space for the egress of egress of excessive blood from the IVD. Alternatively a flap valve or a valve that allows egress of fluid when the fluid exceeds a certain pressure. The catheter could be reinforced with longitudinal fibers embedded in the walls of the catheter. The holes in the tip of the drain could preferably be located on the cranial and caudal sides of the tip as well as the tip of the drain. The holes in the drain could be limited to within 1 to 15 millimeters of the tip of the drain. The tip of the drain could contain a filter that permits fluids, but not cells, to pass through the drain. The pores in such filter are preferably between 8 and 15 microns. Alternatively, such pores could be 5, 6, 7, 16, 17, 18, smaller than 5 or larger than 18 microns in size. Capillary action through such pores preferably aspirates fluids, but not cells, from under the anti-adhesion cover.

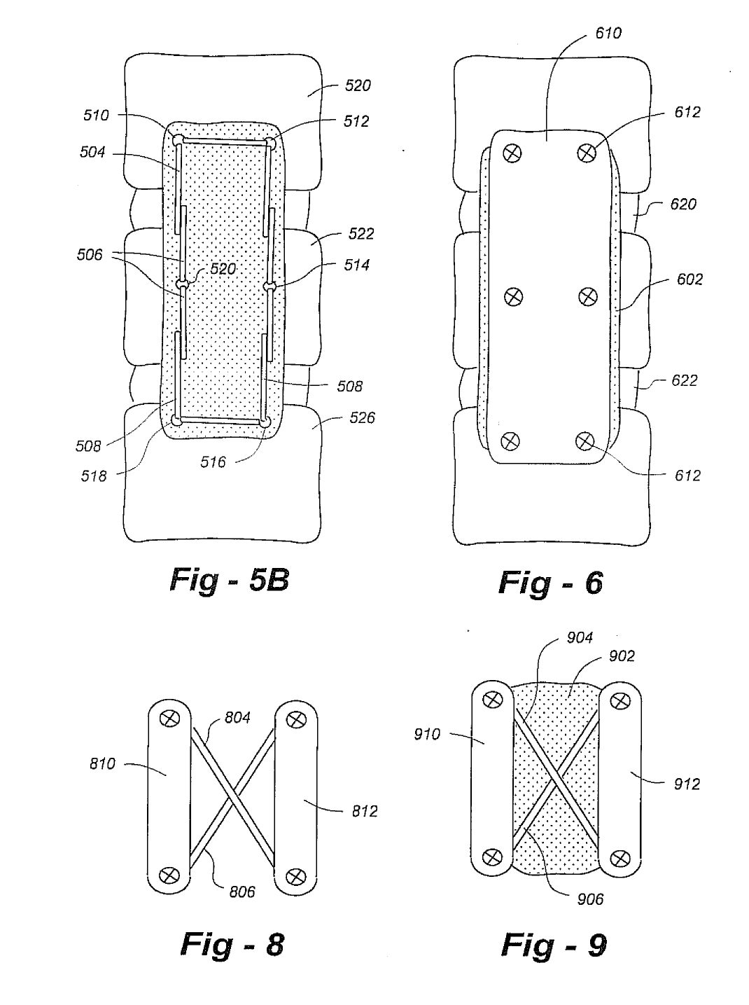

FIG. 5A is an anterior view of a portion of the spine wherein the device drawn in FIG. 2A was applied across two IVDs. The device may be applied across three, four or more IVDs in alternative embodiments of the invention.

FIG. 5B is an anterior view of a portion of the spine and an alternative configuration wherein three flexible longitudinal fixation elements 504, 506, 508 and six suture anchors 510, 512, 514, 516, 518, 520 fasten the anti-adhesion cover to three vertebrae 520, 524, 526 in the configuration shown. The flexible longitudinal fixation elements could be arranged in alternative patterns, such as the cross coupled configuration taught in my co-pending patent applications previously described above, in alternative embodiments of the invention.

FIG. 6 is an anterior view of a portion of the spine, the embodiment of the invention drawn in FIG. 5A and a prior art plate 610 and screws 612. The inventive method uses the anti-adhesion device 602 to seal or nearly seal the IVDs 620, 622 and the plate 610 and screws 612 to immobilize the spine. This prevents hematoma formation outside the IVDs, BMP leakage from the IVDs or disc spaces, or migration of other material into or out of the IVDs and facilitates spinal fusion. The anti-adhesion cover 602 could alternatively be applied over the plate 610 and screws 612. A compressible material such as polyurethane could also be applied to the undersurface of the plate. Such compressible material are preferably closed cell and preferably at least partially cure in-situ. Compressible materials under the plate could form a seal between the plate and the spine thus eliminating the need for an anti-adhesion cover and flexible fixation component in alternative embodiments of the invention.

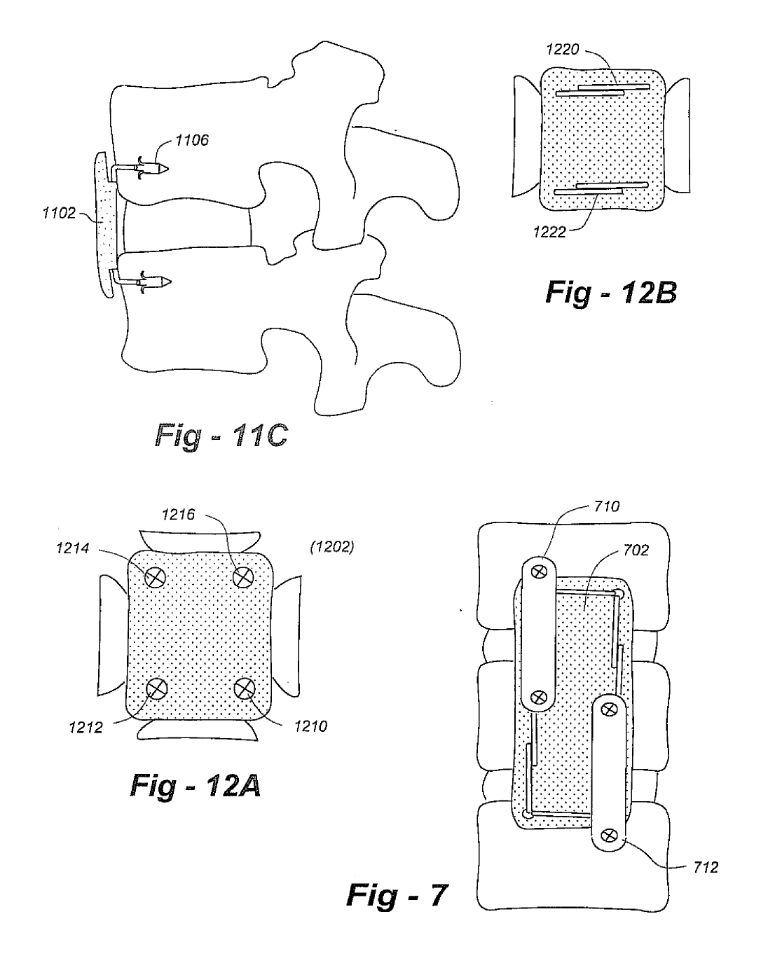

FIG. 7 is an anterior view of a portion of the spine, the embodiment of the invention drawn in FIG. 5A and two prior art plates 710, 712 and screws. The anti-adhesion cover 702 could be applied over the plate and screws in an alternative embodiment of the invention. FIG. 8 is an anterior view of an alternative embodiment of the plates and screws drawn in FIGS. 6 and 7. The plates 810, 812 are connected by flexible longitudinal fixation elements 804, 806 in the configuration shown. FIG. 9 is an anterior view of an alternative embodiment of the invention drawn in FIG. 8. An anti-adhesion cover 902 is fastened to two plates 910, 912 and flexible longitudinal fixation elements 904, 906.

FIG. 10A is an anterior view of a portion of the spine and an alternative embodiment of the invention drawn in FIG. 1A. Suture anchors 1004, 1006 with flexible longitudinal fixation elements 1010, 1012 were inserted into the vertebra above and below an IVD 1020. Suture anchors 1008, 1009 with locking mechanisms were also inserted into the vertebra above and below the IVD. An intra-discal device 1022 is seen in the IVD 1020. Interbody fusion cages, nucleus replacements, total disc replacements, bone graft material or other such intra-discal device or other material may also be placed in the disc space.

FIG. 10B is an anterior view of the portion of the spine, the embodiment of the invention drawn in FIG. 10A and an anti-adhesion cover 1002. The flexible longitudinal fixation elements and suture anchors fasten the anti-adhesion cover to the spine as shown in the drawing.

FIG. 11A is an anterior view of a portion of the spine along with alternative embodiments of the inventions drawn in FIGS. 8F, 8G of my co-pending provisional patent application U.S. Ser. No. 61/118,246 and FIG. 1A of this application. The pores of the anterior and posterior walls of the anti-adhesion sleeve 1102 are preferably less than 10 microns. The posterior surface of the posterior wall of the anti-adhesion cover could have recesses 150 microns or wider to facilitate tissue in-growth. Alternatively, mesh components with pore widths of approximately 1 to 2 millimeters could be adhered to or otherwise fastened to the posterior surface of the posterior wall of the anti-adhesive sleeve. Such mesh components may be made of polyester or polypropylene or other material.

FIG. 11B is an anterior view of the portion of the spine and the embodiment of the invention drawn in FIG. 11A. The anterior wall of the cranial end of the anti-adhesion sleeve was lifted to view two suture anchors 1104, 1106 and portions of two flexible longitudinal fixation elements 1110, 1112. The flexible longitudinal fixation elements pass through the lumen in the anti-adhesion sleeve. FIG. 11C is a lateral view of a partial sagittal cross section of a portion of the spine and the embodiment of the invention drawn in FIG. 11A.

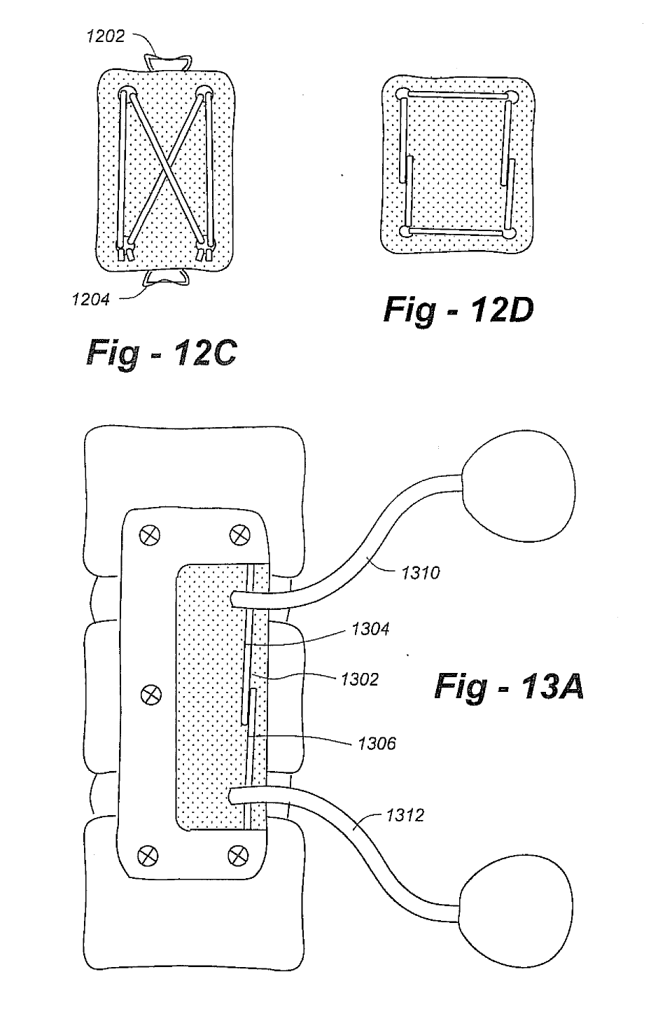

FIG. 12A is an anterior view of an anti-adhesion cover 1202 and prior art methods of fastening such cover to the spine or other area of the body. The fastening elements 1210, 1212, 1214, 1216 are limited to the corners of the anti-adhesion cover. As depicted by the four trapezoid areas in the drawing, such configuration allows blood, fluids, BMP, or other material to egress between the fastening members. Generally the four fastening members obstruct egress of fluids from less than 10% of the periphery of the anti-adhesion cover. Egress of such blood or materials causes adhesions and may lead to compression of structures near the spine. Adding fastening components between along the edge of the anti-adhesion cover using the method taught in FIGS. 1F and 2 of my U.S. Pat. No. 6,371,990 decrease the space for fluids or other materials to egress from the IVD. For example, such additional fastening elements may prevent the egress of fluids from under about 5 to 15% of the periphery of the anti-adhesion cover.

FIG. 12B is an anterior view of the embodiment of the invention drawn in FIG. 9C of my co-pending U.S. patent application Ser. No. 11/946,001. The flexible longitudinal fixation elements 1220, 1222 prevent fluids from leaking from under the cranial and caudal ends of the anti-adhesion cover. Tension on the ends of the flexible longitudinal fixation elements, before fastening the ends to each other presses the anti-adhesion cover against the spine and seals spaces between the cranial and caudal ends of the anti-adhesion cover and the spine. As illustrated in the drawing, egress of fluids or materials is limited to two sides of the anti-adhesion cover. The invention preferably prevents egress of fluids from under at least 10 to 60% of the periphery of the anti-adhesion cover.

FIG. 12C is an anterior view of the embodiment of the invention drawn in FIG. 1A. The configuration of the flexible longitudinal fixation elements and the anti-adhesion cover prevents fluids or other materials from leaking under the sides and most or all of the cranial and caudal sides of the anti-adhesion cover. The drawing illustrates two small areas 1202, 1204 where fluid may egress from the IVD. However, suction from the tip of a drain placed into or near the AF or the disc space, pulls the anti-adhesion cover against the vertebrae and the AF, effectively sealing the area under the anti-adhesion cover. The invention preferably prevents egress of fluids, blood, proteins (such as BMP), or other particles less than 1 mm in width from under at least 60 to 100% of the periphery of the anti-adhesion cover.

FIG. 12D is an anterior view of the embodiment of the invention drawn in FIG. 2A. The device prevents or substantially prevents fluids or other materials from leaking from through anulotomies (surgical defects) in the AF. The invention preferably prevents egress of fluids, blood, proteins (such as BMP), or other particles less than 1 mm in width from under at least 90 to 100% of the periphery of the anti-adhesion cover.

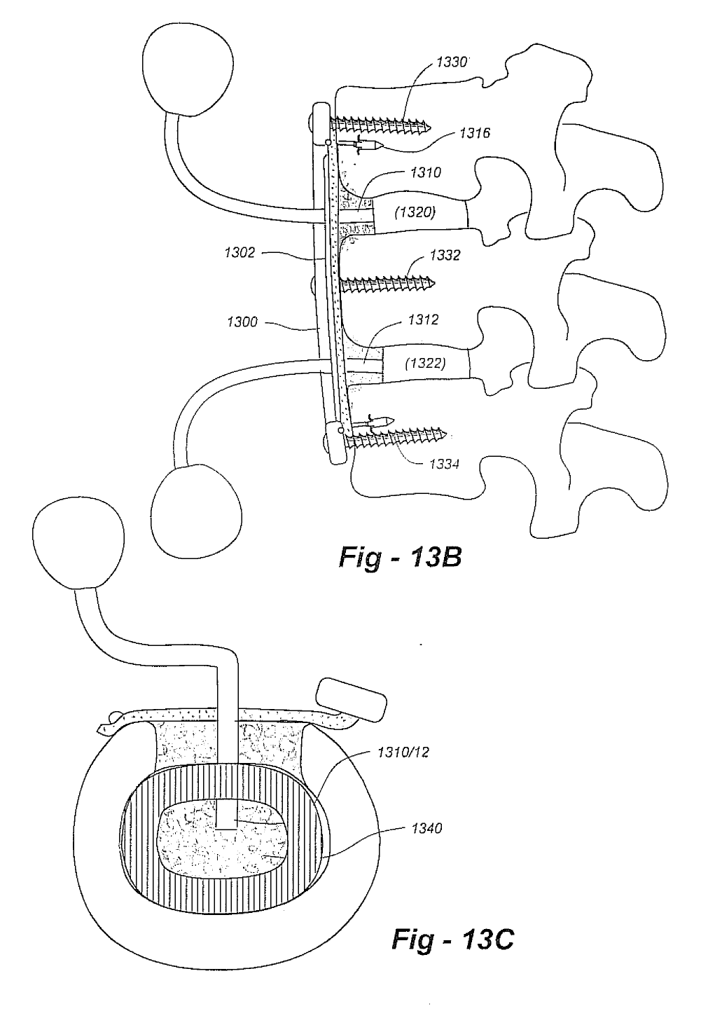

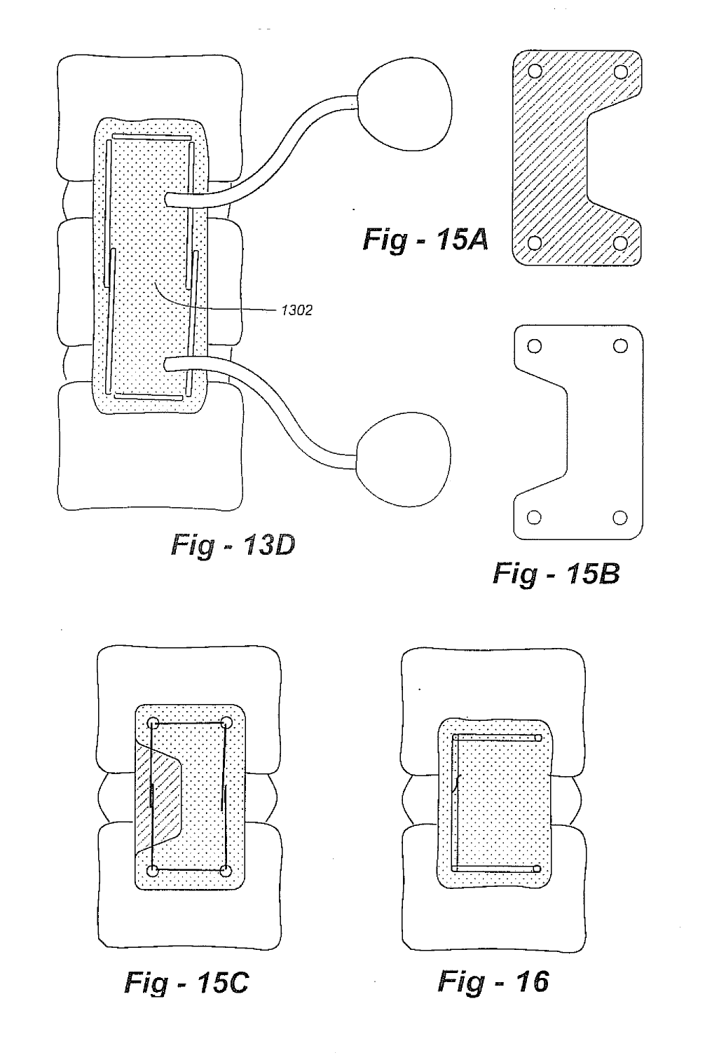

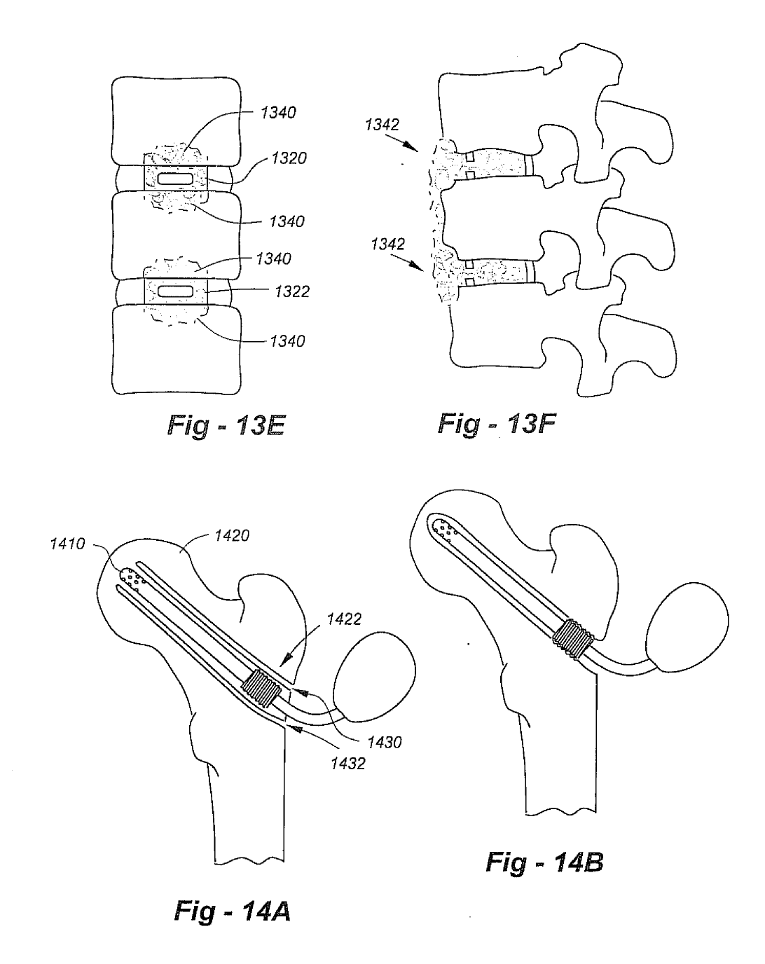

FIG. 13A is an anterior view of a portion of the spine and an alternative embodiment of the invention drawn in FIG. 6. A novel C-shaped plate 1300 is seen over the anti-adhesion cover 1302 and flexible longitudinal fixation elements 1304, 1306. The tips of two drains 1310, 1312 extend through openings in the anti-adhesion cover and into two disc spaces. A tight fit between the catheters of the drains and the anti-adhesion cover prevents blood, fluids, proteins, or other material from passing through such locations.

FIG. 13B is a lateral view of a partial sagittal cross section of the portion of the spine and embodiment of the invention drawn in FIG. 13B. The tips of the drains 1310, 1312 extend through openings in the anterior portions of two interbody cages 1320, 1322. Bone growth material is seen anterior to the interbody cages and anterior to portions of the vertebrae. The drawing illustrates two push-in anchors 1316, 1318 and three screws 1330, 1332, 1334 which fasten the flexible longitudinal fixation elements and the plate 1300 to the spine, respectively. The screws 1330, 1332, 1334 are placed cranial or caudal to the horizontal arms of the flexible longitudinal fixation elements and medial or lateral to the vertical arms of such flexible fixation elements to avoid damaging such flexible components. The tips of the drains 1310, 1312 are seen passing through the anti-adhesion cover 1302, into the disc spaces, and into the interbody cages.

FIG. 13C is a view of an axial cross section of an IVD and the embodiment of the invention drawn in FIG. 13B. The tip of the drain extends through an opening in the anterior portion of the interbody cage and into the center of the cranial to caudal opening in such cage. As described in FIG. 5A, of my co-pending patent application Ser. No. 11/811,751, the flexible longitudinal fixation elements and the anchors lie medial, lateral, cranial, and caudal the aperture in the AF to seal such aperture. Bone growth material 1340 is seen within and anterior to the interbody cage. The tip of the drain may be placed anterior to the cage or within the opening in the anterior portion of the cage in alternative embodiments of the invention. The distal tip of the flexible drain could be enlarged to help prevent inadvertent, premature withdraw of the drain. The enlarged tip is preferably collapsible to enable the tip to be pulled through the opening in the anti-adhesion cover.

FIG. 13D is an anterior view of a portion of the spine and the embodiment of the invention drawn in FIG. 13A. The plate was not included in the drawing to better illustrate the anti-adhesion cover 1302. FIG. 13E is an anterior view of a portion of the spine and the embodiment of the invention drawn in FIG. 13D. The anti-adhesion cover and flexible longitudinal fixation components were not included to better illustrate the interbody cages 1320, 1322 and the vertebrae. Bone growth material 1340 is seen within openings in the cages and anterior to the vertebrae.

FIG. 13F is a lateral view of a partial sagittal cross section of the spine and the embodiment of the invention drawn in FIG. 13E. Unlike prior-art fusion procedures, bone growth material 1342 is seen anterior to the cages and the vertebrae. The anti-adhesion holds such bone growth material in such position. Since the anti-adhesion cover is impervious to blood and cells, such as mesenchymal stem cells (MSCs), the invention contains or substantially contains such blood and MSCs and the anti-adhesion cover preferably directs MSCs to a preferred fusion area. For example, the invention may direct MSCs from decorticated anterior surfaces of the vertebrae to bone growth material in the disc spaces. The entire anterior surface of one or more vertebrae or anterior portions of such vertebrae may be decorticated to maximize the surface for MSCs migration from the vertebrae. A trough could be machined into the anterior surface of one or more vertebrae that are covered by the anti-adhesion cover. Such trough could be filled with bone growth material. The invention may increase the fusion area by 200% or more compared to prior art fusion procedures.

FIG. 14A is an anterior view of a partial coronal cross section of the proximal end of the femur and an alternative embodiment of the invention. The tip of a drain 1410 is seen in the femoral head 1420. The catheter of the drain extends through a cannulated screw component 1422 and outside the femur. The cannulated screw seals the area around the catheter of the drain. One, two or more holes 1430, 1432 were drilled parallel to the hole that contains the catheter of the drain. Suction from the drain pulls blood that contains MSCs from a more distal location in the femur to the head of the femur. The invention may be used to treat avascular necrosis of the hip. The drain is preferable inserted through a cannula that is preferably inserted into a hole drilled in the femur. Such method prevents clogging the pores of the drain as the drain is inserted into the bone.

FIG. 14B is an anterior view of a partial coronal cross section of the proximal end of the femur and an alternative embodiment of the invention drawn in FIG. 14A. The catheter was placed into a hole wider than the diameter of the drain. As described in FIG. 14A, the invention causes blood and MSCs to migrate from a more distal location in the bone to a more proximal location. The invention may be used in other areas of the body. For example, the invention could be used to fuse or stimulate healing of fractures of bones such as bones in the foot, ankle, leg, pelvis, skull or arm. The invention taught in this patent application aspirates blood that contains MSCs from one or more bones into another area of such bones or other areas of the body and removes MSC poor blood or fluid from such bones or areas. The invention facilitates tissue healing by concentrating MSCs in the desired area. A seal between the catheter the area where MSCs will be concentrated and the surrounding tissues enables aspiration of blood and captures MSCs in the desired areas. The invention increases the numbers of MSCs and concentrates the MSCs within the surgical area while limiting hematoma size and location. The invention facilitates healing and reduces complications.

FIG. 15A is an anterior view of an alternative anti-adhesion cover of an alternative embodiment of the invention drawn in FIG. 4. FIG. 15B is an anterior view of a second anti-adhesion cover used in the embodiment of the invention drawn in FIG. 15A. FIG. 15C is an anterior view of a spinal segment and the anti-adhesion covers drawn in FIGS. 15A and 15B. The overlapping anti-adhesion covers form a flap valve that releases excessive fluids from the disc space. The covers are fastened to the spine with suture anchors and flexible longitudinal fixation elements.

FIG. 16 is an anterior view of a spinal segment and an alternative embodiment of the invention drawn in FIG. 15C. The flexible longitudinal fixation components are limited to three sides of the anti-adhesion cover. The opening under the fourth side of the anti-adhesion cover permits excessive fluid to leak from the disc space. The suture anchors on the left side of the drawing contain two flexible longitudinal fixation elements. The suture anchors on the right side of the drawing contain one flexible longitudinal fixation element. Two, three, or four sides of the anti-adhesion cover could permit excessive fluid to escape in alternative embodiments of the invention.

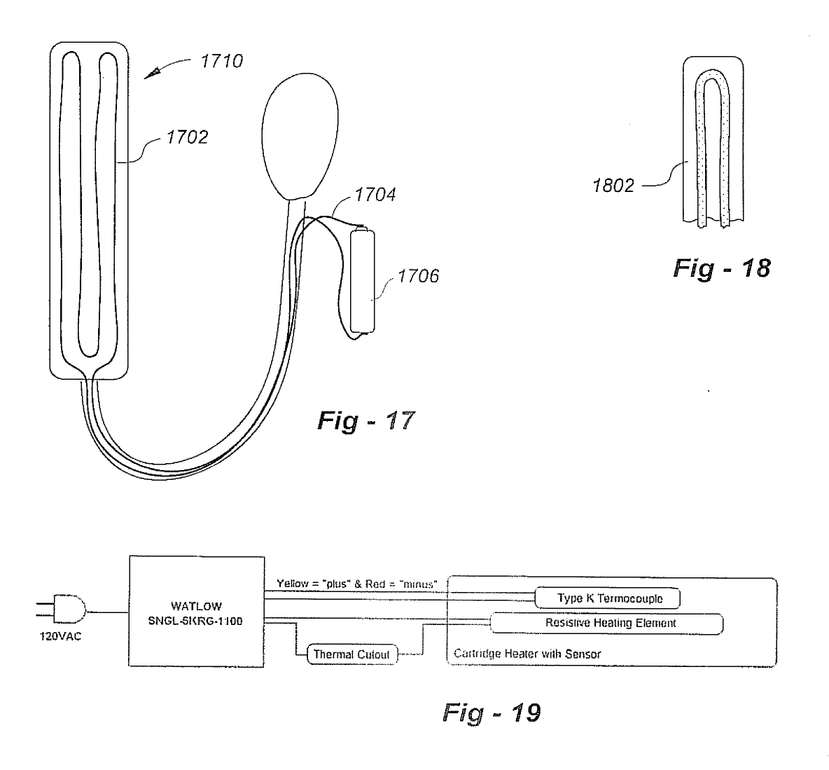

FIG. 17 is an anterior view of an alternative embodiment of the invention drawn in FIG. 4. The drain includes a thermal element 1702. For example, such thermal element could be a wire 1704 that the passes from a battery 1706 or other source of power to the distal tip 1710 of the drain. The thermal element preferably heats the distal portion of the drain and the surrounding tissues to temperatures in the range of 99 to 110° F. Most preferably the distal end of the drain and the surrounding tissues are heated to temperatures in the range of 2 to 4° F. above core body temperature. For example, in humans such drain would most preferably be heated to 100 to 104° F. Alternatively, the drain could be heated to more than 110° F., especially if such heated drain is used in animals whose core body temperature is higher than humans' core body temperature. In all embodiments, temperature controllers known to those skilled in electrical apparatus would be used to maintain the heating element(s) within a desired temperature range.

The heating element may be coated or molded in polypropylene, polyethylene, silicon, silastic or other biocompatible material with or without a lumen to drain fluid. In preferred embodiments, the heating element is provided in the form of an elongated, flexible catheter-like sheath having an overall length of 10-40 cm, more or less, which is thermally insulated except for the heated tip portion which may be 1-8 or more preferably 2-6 cm in length. A coated or uncoated wire may form the heating element itself or, as discussed below, a heated fluid may be circulated through the distal tip portion.

The distal wire portion of the heating element is preferably activated at the completion of the surgical procedure and remains in the patient for 1 to 7 days following surgery. Most preferably the wire of the heating element remains in patients 2 to 4 days following surgery. Alternatively, such wire element could remain in the patient for 8 to 14 days, or longer. The battery or power source component of the invention is preferably outside a patient's or an animal's skin. The heating element may be pulled from the patient or animal after heating the tissues.

The invention broadly raises the temperature of the surgical tissues to stimulate inflammation, thereby stimulating tissue healing. Unlike prior art technology, which places heat elements on the skin, the invention places the heat element below the skin and adjacent to the tissue stimulated to heal. The heat from prior art external devices is concentrated on the skin and the tissues near the skin rather than on the deeper tissues. Thus, peripheral heating elements may increase inflammation and attract MSCs to the peripheral tissues rather than to the deeper tissues. In fact such peripheral heating elements may cause fewer MSCs and fewer inflammatory cells to migrate into the deeper tissues. Furthermore, the blood flow through the skin and muscles between peripheral heating elements and the deeper tissues shunts the heat away from the heated area. Thus, external heating elements require substantially higher temperatures, which may burn patients' skin, to raise the temperature of the deeper tissues by 1° to 2° F., compared to the invention which places heat elements in deeper tissues.

The invention enables heating of tissues 1 to 25 centimeters or more below the surface of the skin. The invention may be used to stimulate or enhance the healing of any injured tissue. For example, heating elements could be temporarily placed against relatively avascular tissues such as the intervertebral disc, ligaments, meniscus of the knee, articular cartilage or other such tissue to stimulate healing of injuries to such tissues. Alternatively, heating elements could be placed through blood vessels and directed into the heart, to increase the temperature of the blood within the heart and increase the temperature of the heart to stimulate healing of myocardial tissue after myocardial infarction (MI). Such cardiac or blood vessel heating elements are preferably used to heat the heart within a few hours of MI. For example, the heart could be heat to 1° to 4° F. within six hours of MI and heated for 48 hours after MI. Alternatively, the heart could be heated by less than 1° F. or more than 4° F. for 10, 15, 20, 25, 30, 35, 40, 45, 50 hours or less than 10 hours or more than 50 hours.

The invention could also be used to stimulate bone growth. For example, the heat element could be placed adjacent to vertebrae and bone growth material in spinal fusion operations. The invention attracts MSCs to the vertebrae and bone growth material within hours of surgery. Alternatively, the invention could be used to stimulate bone growth into prosthetic joints. For example, heating elements could be used to increase the temperature of bone in-growth artificial joints of the hip, knee, shoulder, or other joint by 1° to 4° F. for 1 to 72 hours, or longer following surgery. The invention could also be used to stimulate healing of fractured bones. The invention could be used to stimulate or enhance healing of any tissue in humans or other animals. The invention may also be used to protect cells in injured tissues in humans or animals.

This aspect of the invention is designed to accelerate and intensify the inflammatory phase of healing. Thus, the heating element should be activated at the end of the surgical procedure and should likely be removed a few days following the procedure. Heating the tissues during the proliferation and resolution or remodeling phases of healing could be counter-productive. Alternatively, healing could be continued for more than 2 weeks and into the phases following the inflammation phase of healing. If such heating device is continued beyond 1 to 2 weeks of surgery, the entire heating device, including the battery, is preferably implanted in the patient. Such device could be surgically removed 1, 2 or more weeks following surgery. Heating tissues may also reduce the risk of infections. The invention may be used to stimulate healing and prevent or treat infection in any tissue of any human or animal.

FIG. 18 is an anterior view of the distal portion of an alternative embodiment of the invention drawn in FIG. 17. Heated fluid 1802, such as saline, is circulated through the lumen or lumens of one or more loops within the distal portion of the invention. The heated fluid preferably heats the surrounding tissues to the temperatures described in the text relating to FIG. 17. Alternatively, cooled fluid could be circulated through the element to reduce inflammation to protect injured neurological tissues such as the brain and spinal cord, or other injured tissues. Alternatively, the tissues could be heated to the previously mentioned temperatures by other means in alternative embodiments of the invention. For example, heating coils could temporarily placed in or near blood vessels, preferably arteries, that supply the surgically treated tissues. In any case the devices used to heat or cool the fluid would preferably be disposed outside the body and circulated with a peristaltic or other pumping mechanism. Alternatively, an external device for example one that emits infrared light, could be used to temporarily heat the surgically treated tissues.

FIG. 19 is schematic of a preferred embodiment of the invention. Watlow Firerods, Thermocouples, and Single Channel Temperature Control Panels are available from OEM Supply Inc, Swansea Mass. High-Temperature Cartridge Heaters are available from McMaster Carr, Cleveland Ohio. A Cantherm SDJ1 DFF077S thermal cutoff (Cantherm, Montreal Quebec, Canada) is preferably added in line with the power fed to the heater. The resistive heating element is placed into, adjacent to, or near the tissues to be stimulated.

1. Apparatus for heating internal human or animal tissue, comprising:
an elongated, biocompatible member having a distal tip adapted for implantation within a body adjacent to tissue;
a heating element disposed at the distal tip; and
a source of power operative to maintain the temperature of the heating element and the tissue at a few degrees above body temperature.

2. The apparatus of claim 1, wherein:
the heating element is an electrically conductive wire; and
the source of power is a battery connected to the wire.

3. The apparatus of claim 1, wherein:
the heating element is an electrically conductive wire;
the source of power is a battery connected to the wire; and
the battery is also adapted for implantation within the body.

4. The apparatus of claim 1, wherein:
the heating element includes a heated fluid; and
the source of power includes a fluid heater and pump to circulate the fluid through the distal tip.

5. The apparatus of claim 1, wherein the elongated, biocompatible member is thermally insulated along its length with the exception of the heated distal tip.

6. A method of promoting healing, comprising the steps of:
providing the apparatus of claim 1; and
positioning the heated distal tip 1 to 25 centimeters or more below the surface of the skin of a recipient.

7. A method of promoting healing, comprising the steps of:
providing the apparatus of claim 1; and
positioning the heated, distal tip adjacent an injured intervertebral disc, ligament, meniscus, articular cartilage or other tissue to stimulate the healing thereof.

8. A method of promoting healing, comprising the steps of:
providing the apparatus of claim 1; and
positioning the heated, distal tip through a blood vessel and into the heart to increase the temperature of the blood within the heart and stimulate the healing of myocardial tissue following a myocardial infarction (MI).

9. A method of promoting healing, comprising the steps of:
providing the apparatus of claim 1; and
positioning the heated, distal tip adjacent a bone or vertebral body to stimulate bone growth following a spinal fusion operation.

10. A method of promoting healing, comprising the steps of:
providing the apparatus of claim 1; and
positioning the heated, distal tip into a prosthetic joint to stimulate bone in-growth associated with an artificial hip, knee, shoulder, or other joint.

11. A method of promoting healing, comprising the steps of:
providing the apparatus of claim 1; and
positioning the heated, distal tip near arteries or other blood vessels that supply surgically treated tissues.

12. A method of promoting healing, comprising the steps of:
providing the apparatus of claim 1; and
implanting the apparatus for a period of 1 to 72 hours following a surgical operation.
